(19) United States
(12) Patent Application Publication
(10) Pub. No.: US 2009/0204104 A1
(43) Pub. Date: Aug. 13, 2009

(54) Methods for Compounding and Delivering a Therapeutic Agent to the Adventitia of a Vessel

(75) Inventors: Patrice Tremble, Santa Rosa, CA (US); Dianne Judd, Minneapolis, MN (US); Kishore Udipi, Santa Rosa, CA (US); Peiwen Cheng, Santa Rosa, CA (US); Rangarajan Sundar, Santa Rosa, CA (US); Kaushik A. Patel, Windsor, CA (US); Eugene Tedeschi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc, Santa Rosa, CA (US)

(21) Appl. No.: 11/568,779

(22) PCT Filed: May 4, 2005
(86) PCT No.: PCT/US05/15621

§ 371 Date: Nov. 7, 2006

Related U.S. Application Data
(60) Provisional application No. 60/570,691, filed on May 13, 2004.

(51) Int. Cl. A61M 25/10; A61M 25/06; A61K 9/14; A61P 9/02

(52) U.S. Cl. 604/509; 604/508; 424/489

(57) ABSTRACT

The invention provides a method of delivering a therapeutic agent to the adventitia of a vessel using a catheter-based microsyringe. A therapeutic agent is formed into microparticles, which are dispersed throughout an appropriate liquid carrier to form a therapeutic mixture. A catheter is provided that includes a microsyringe operably attached to an actuator. The microsyringe includes a hollow needle in fluid communication with a therapeutic agent delivery conduit. The catheter is introduced into a target area of a vessel. The actuator is operated to thrust the needle into a wall of the vessel. The therapeutic mixture is supplied to the therapeutic agent delivery conduit and delivered through the conduit to the needle and thereby into the adventitia of the vessel. The actuator is again operated to withdraw the needle from the wall of the vessel and to enclose it within the actuator. The catheter is then removed from the vessel.

### TECHNICAL FIELD

This invention relates generally to treatment of vascular conditions. More specifically, the invention relates to delivery of a therapeutic agent to the adventitia of a vessel for treatment of a vascular condition.

### BACKGROUND OF THE INVENTION

Heart disease, specifically coronary artery disease, is a major cause of death, disability, and healthcare expense in the United States and other industrialized countries. A wide variety of methods have been developed to provide treatment to diseased coronary arteries.

Studies have shown that delivering a therapeutic agent into the outer membrane of an artery, termed the tunica adventitia, adventitial layer, or simply adventitia, allows the agent to permeate the vessel. Thus the adventitia is capable of acting as a circulatory system within and for the artery.

A therapeutic agent may be delivered to the adventitia through the outer wall of the vessel by percutaneous injection or through the inner wall of the vessel by catheter delivery. For example, U.S. Patent Application Publication No. 2002/0022055 discloses a method for improving or increasing body passageway or cavity integrity that includes percutaneous delivery of a therapeutic agent by direct injection via an outer wall of the body passageway or cavity into the adventitia. The method includes applying a polymer or therapeutic agent/polymer complex to the external portion of the vessel as a periadventitial wrap. U.S. Patent Application Publication No. 2003/0077279 discloses a method for treating vascular disease by inhibiting toll-like receptor-4 (TLR-4). The method includes delivering a TLR-4 inhibiting composition by coating the composition onto a stent or by injecting the composition into the media and inner adventitia using an intravascular catheter.

Vascular delivery of therapeutic agents can be accomplished using a number of different delivery methods and devices. One such device is disclosed in U.S. Patent Application Publication No. 2003/0055446. This device includes an actuator joined to a distal end of a catheter. The actuator includes an expandable section designed to deploy a needle. When the expandable section is in an unactuated, furled condition, the needle is enclosed within the folds of the expandable section, preventing the needle from injuring the vessel walls while the catheter is being introduced into the target area of a vessel. Fluid connections are provided at the distal end of the catheter and a proximal end of the actuator to supply a therapeutic or diagnostic agent to the needle and to provide an activating fluid to the actuator. When actuated, the expandable section unfurls and expands, thrusting the needle outward and into a position approximately perpendicular to the vessel wall, thereby penetrating the vessel wall. When the activating fluid is removed, the expandable section returns to a furled state with the needle again enclosed within the folds of the expandable section to prevent trauma to the vessel during removal of the catheter.

While many therapeutic agents and their liquid carriers are suitable for delivery into the adventitia, many more are not. Because the adventitia is primarily fat and elastic fibers, lipophilic agents are particularly well absorbed and distributed. However, lipophilic agents typically require organic solvents that can cause cytotoxicity, hypersensitivity reactions, and other undesirable effects when delivered directly into tissue. Even therapeutic agents that are carried in a nontoxic fluid may damage the tissue of a vessel if their release is not controlled to prevent toxic levels accumulating within the tissue. Therefore, it would be desirable to have methods for compounding and delivering a therapeutic agent to the adventitia of a vessel that overcome the aforementioned and other disadvantages.

### SUMMARY OF THE INVENTION

One aspect of the present invention is a method of delivering a therapeutic agent to the adventitia of a vessel using a catheter-based microsyringe. A therapeutic agent is formed into a plurality of microparticles. The microparticles are dispersed without dissolving throughout a pharmaceutically acceptable liquid carrier to form a therapeutic mixture or emulsion. A catheter is provided. The catheter includes a microsyringe operably attached to an actuator. The microsyringe includes a hollow needle in fluid communication with a therapeutic agent delivery conduit. The actuator is operable between an unactuated condition in which the needle is enclosed within the actuator and an actuated condition in which the needle is thrust outward by the actuator. The catheter is introduced into a target area of a vessel. The actuator is operated such that the needle is thrust outward and into a wall of the vessel. The therapeutic mixture is supplied to the therapeutic agent delivery conduit and delivered through the conduit to the needle and through the needle into the adventitia of the vessel. The actuator is operated such that the needle is withdrawn from the wall of the vessel and again enclosed within the actuator. The catheter is then removed from the vessel.

Another aspect of the present invention is a method of compounding a therapeutic agent for delivery to the adventitia of a vessel using a catheter-based microsyringe. A therapeutic agent is formed into a plurality of microparticles. The microparticles are dispersed throughout a liquid carrier suitable for delivery to the adventitia of a vessel.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is a flow diagram of one embodiment of a method of delivering a therapeutic agent to the adventitia of a vessel using a catheter-based microsyringe, in accordance with the present invention;

FIG. 2 is a schematic, perspective view of a catheter-based microsyringe in accordance with the present invention;

FIG. 3 is a transverse cross-section of an artery, showing the adventitia of the artery;

FIG. 4 is a flow diagram of one embodiment of a method of compounding a therapeutic agent for delivery to the adventitia of a vessel using a catheter-based microsyringe, in accordance with the present invention.

### DETAILED DESCRIPTION OF THE PRESENTLY PREFERRED EMBODIMENTS

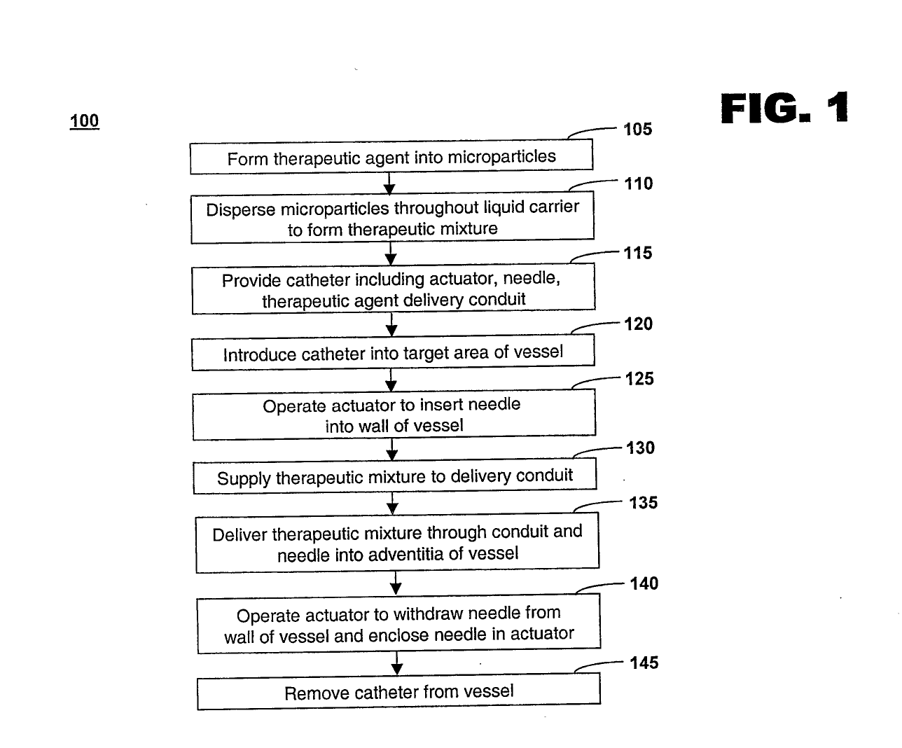

One aspect of the present invention is a method of delivering a therapeutic agent to the adventitia of a vessel using a catheter-based microsyringe. FIG. 1 shows a flow diagram of one embodiment of the method at 100, in accordance with the present invention.

A therapeutic agent is formed into a plurality of microparticles (Block 105). The therapeutic agent may include, for example, an antiproliferative agent, an antineoplastic agent, an antibiotic agent, an anti-inflammatory agent, an angiogenesis inhibitor, a metalloproteinase inhibitor, a serine proteinase inhibitor, molecules that block adhesion of lymphocytes or other immune response cells, combinations thereof, and the like. Microparticles of this invention may be nanoparticles or larger, e.g. up to 50 μm in diameter.

The microparticles may be formed by, for example, combining the therapeutic agent with a plurality of microspheres. The therapeutic agent may be encapsulated within the microspheres or attached to the outer surface of the microspheres, both techniques being known in the art. The microspheres may comprise either a biodegradable or a biocompatible matrix material. The matrix material may be a biodegradable polymer such as polylactide (PLA) or a biocompatible polymer such as a cellulose-based polymer. The matrix material may also be a protein such as albumin, a polysaccharide such as dextrans, or a lipid such as stearic acid. Where the matrix material is a lipid, the microsphere may be termed a liposome, i.e., a spherical particle formed by a lipid bilayer enclosing an aqueous compartment.

Alternatively, the therapeutic agent may be formed into dendrimers or carried as guest molecules within dendritic voids. A dendrimer is an artificially manufactured or synthesized molecule built up from branched monomers. Dendrimers have two major chemical environments: the surface chemistry due to the functional groups on the termination generation, which is the surface of the dendritic sphere; and the sphere's interior, the dendritic void, which is largely shielded from exterior environments due to the spherical shape of the dendrimer structure. Dendrimer research has confirmed the ability of dendrimers to accept guest molecules in the dendritic voids.

In yet another alternative, the microparticles may be formed into globules that comprise the discontinuous phase of an emulsion. By definition, the discontinuous phase of an emulsion is the dispersed liquid, and the continuous phase is the dispersion medium. In pharmaceutical preparations, forming a therapeutic agent into globules may include coating the globules with a gum or other mucilaginous substance.

The microparticles (regardless of how formed) are dispersed without dissolving into a pharmaceutically acceptable liquid carrier to form a therapeutic emulsion or mixture (Block 110). For a liquid carrier to be pharmaceutically acceptable for the present invention, it must be capable of being delivered directly into the adventitia without causing cytotoxicity, hypersensitivity reactions, or other undesirable effects. For example, a saline or other aqueous solution or, for an emulsion, a biocompatible dispersion medium would be an acceptable carrier. Organic solvents such as Cremaphor and ethanol would not be acceptable for the present invention.

Forming the therapeutic agent into microparticles permits some therapeutic agents, for example those that are lipophilic, to be dispersed throughout a nontoxic carrier fluid that is not otherwise a solvent for the therapeutic agent itself. Timed release in a highly controlled manner may also be facilitated by forming the therapeutic agent into microparticles. For example, where a therapeutic agent has been encapsulated within microspheres, the agent may be released over an extended period of time as a biodegradable polymer used as a matrix for the microspheres erodes or otherwise degrades, providing a continuous release of the agent while preventing it from reaching toxic levels. The agent may also be timed for release after a predetermined delay. Such timed release may be especially useful where the agent is a moderate to highly lipophilic or a hydrophilic therapeutic agent.

A catheter is provided (Block 115). The catheter includes a microsyringe, a microsyringe being a device for ejecting liquids through a small aperture. The microsyringe is operably attached to an actuator. The microsyringe includes a hollow needle in fluid communication with a therapeutic agent delivery conduit.

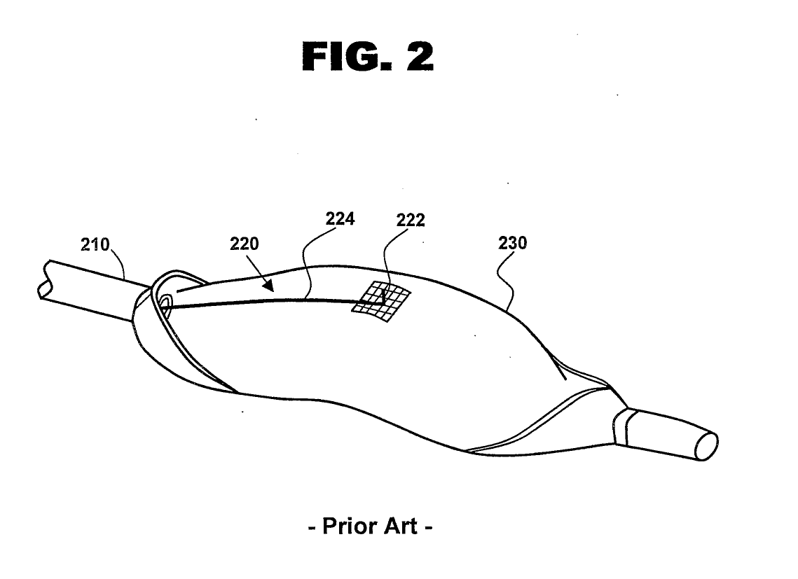

FIG. 2 shows a schematic, perspective view of a catheter-based microsyringe which may be used in accordance with the present invention. Catheter 210 includes a microsyringe 220 operably attached to an actuator 230. Microsyringe 220 includes a hollow needle 222 in fluid communication with a therapeutic agent delivery conduit 224. The actuator is positioned on a distal portion of the catheter and may comprise an inflatable balloon, as shown in this illustration. The present embodiment is not limited to a particular microsyringe; however, the invention is especially useful with the device disclosed in U.S. Patent Application Publication No. 2003/0055446 A1.

The needle and delivery conduit are shown in FIG. 2 as generally straight bodies. However, where the therapeutic mixture to be delivered is an emulsion, it may be desirable for a portion of either or both of the needle and the therapeutic agent delivery conduit to be tortuous, resulting in turbulence that mixes the emulsion prior to delivery. The emulsion may also be mixed prior to delivery by turbulence in the delivery conduit produced by two or more channels emptying into a single lumen within the delivery conduit.

The catheter is introduced into a target area of a vessel (Block 120). For example, a percutaneous access site may be created in the vessel to be treated or a vessel that leads to the vessel to be treated. A guidewire or a guiding catheter may be introduced through the percutaneous access site and advanced to a position adjacent to the target area of the vessel. The catheter including the microsyringe may be introduced into the vessel, either over a guidewire or directly into the guiding catheter. The catheter may then be guided to the target area of the vessel.

Once the catheter is in place, the actuator is operated to thrust the needle outward and into the wall of the vessel (Block 125). Where the actuator comprises an inflatable balloon, the act of inflating the balloon may move the needle into a position approximately perpendicular to the vessel wall, thereby thrusting the needle outward and inserting it into the vessel wall. The balloon is shown in FIG. 2 in an actuated condition. Prior to operating the actuator, the needle may be held inside the folds of the uninflated balloon, preventing the needle from injuring the vascular walls while the catheter is being introduced into the target area of the vessel.

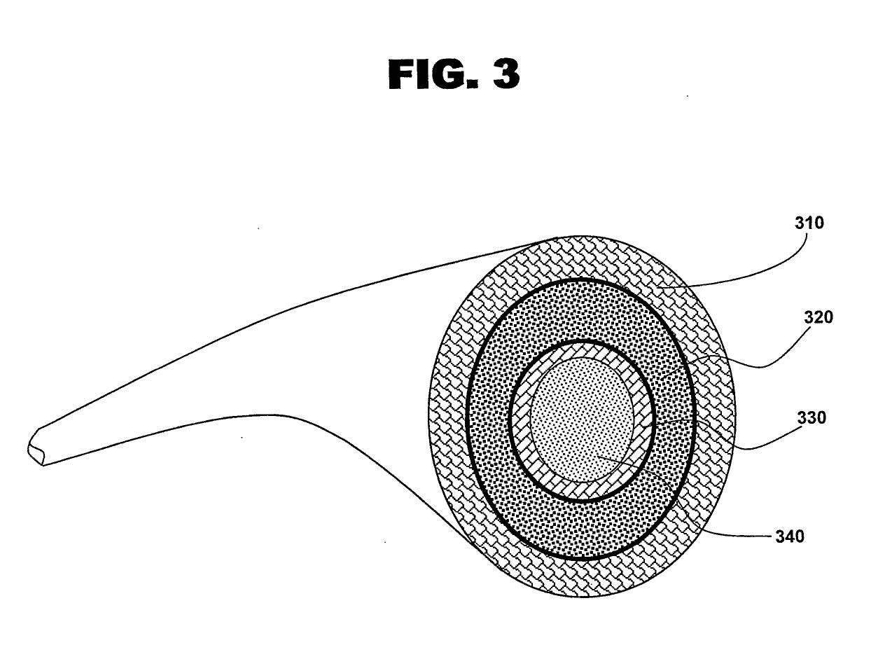

The therapeutic mixture is supplied to the therapeutic agent delivery conduit from, for example, a reservoir positioned outside the body of the individual undergoing therapy (Block 130). The therapeutic mixture is then delivered through the conduit into the needle and through the needle into the adventitia of the vessel (Block 135). FIG. 3 is an illustration of an artery with the adventitia (also referred to as tunica adventitia or outer membrane) of the vessel indicated at 310. The tunica media, tunica intima, and lumen are indicated at 320, 330, and 340, respectively. Studies have shown that delivering a therapeutic agent into the adventitia, which comprises fat and elastic fibers, allows the agent to permeate the vessel.

After delivery of the therapeutic agent into the adventitia, the actuator is operated to withdraw the needle from the wall of the vessel and enclose it within the actuator (Block 140). Where the actuator comprises an inflatable balloon, deflating the balloon may move the needle back inside the folds of the deflated balloon, thereby enclosing the needle and preventing trauma to the vessel during removal of the catheter from the vessel. The catheter may then be removed from the vessel (Block 145). As will be clear to one skilled in the art, the catheter may be repositioned and the steps for delivering the therapeutic agent to the adventitia repeated any number of times before removing the catheter from the vessel.

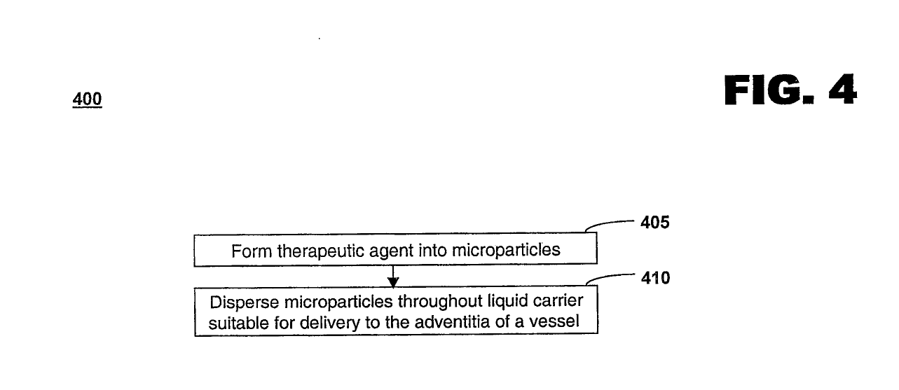

Another aspect of the present invention is a method of compounding a therapeutic agent for delivery to the adventitia of a vessel using a catheter-based microsyringe. One embodiment of the method, in accordance with the present invention, is diagrammed in FIG. 4 at 400.

A therapeutic agent is formed into a plurality of microparticles (Block 405). The therapeutic agent may include, for example, an antiproliferative agent, an antineoplastic agent, an antibiotic agent, an anti-inflammatory agent, an angiogenesis inhibitor, a metalloproteinase inhibitor, a serine proteinase inhibitor, molecules that block adhesion of lymphocytes or other immune response cells, combinations thereof, and the like. Microparticles of this invention may be nanoparticles or larger, e.g. up to 50 μm in diameter.

The microparticles may be formed by, for example, combining the therapeutic agent with a plurality of microspheres. The therapeutic agent may be encapsulated within the microspheres or attached to the outer surface of the microspheres, both techniques being known in the art. The microspheres may comprise either a biodegradable or a biocompatible matrix material. The matrix material may be a biodegradable polymer such as polylactide (PLA) or a biocompatible polymer such as a cellulose-based polymer, for example ethyl cellulose, carboxymethylcellulose, cellulose acetate, methylcellulose or any other acceptable polymer. The matrix material may also be a protein such as albumin, a polysaccharide such as dextrans, or a lipid. Where the matrix material is a lipid, the microsphere may be termed a liposome, a spherical particle formed by a lipid bilayer enclosing an aqueous compartment.

Alternatively, the therapeutic agent may be formed into dendrimers or carried as guest molecules within dendritic voids. A dendrimer is an artificially manufactured or synthesized molecule built up from branched monomers. Dendrimers have two major chemical environments: the surface chemistry due to the functional groups on the termination generation, which is the surface of the dendritic sphere; and the sphere's interior, the dendritic void, which is largely shielded from exterior environments due to the spherical shape of the dendrimer structure. Dendrimer research has confirmed the ability of dendrimers to accept guest molecules in the dendritic voids.

In yet another alternative, the microparticles may be formed into globules that comprise the discontinuous phase of an emulsion. By definition, the discontinuous phase of an emulsion is the dispersed liquid, and the continuous phase is the dispersion medium. In pharmaceutical preparations, forming a therapeutic agent into globules may include coating the globules with a gum or other mucilaginous substance, including, without limitation, xanthan gum, carrageenan, gum arabic, guar gum.

The microparticles are dispersed throughout a liquid carrier suitable for delivery to the adventitia of a vessel (Block 410). For a liquid carrier to be suitable, it must be capable of being delivered directly into the adventitia without causing cytotoxicity, hypersensitivity reactions, or other undesirable effects. For example, a saline or other aqueous solution or, for an emulsion, a biocompatible dispersion medium would be an acceptable carrier. Organic solvents such as Cremaphor and ethanol would not be suitable for the present invention.

Forming the therapeutic agent into microparticles permits some therapeutic agents, for example those that are lipophilic, to be dispersed throughout a nontoxic carrier fluid that is not otherwise a solvent for the therapeutic agent. Timed release of a lipophilic or a hydrophilic therapeutic agent may also be achieved by forming the therapeutic agent into microparticles. For example, where a therapeutic agent has been encapsulated within microspheres, the agent may be released over an extended period of time as a biodegradable polymer used as a matrix for the microspheres erodes or otherwise degrades, providing a continuous release of the agent while preventing it from reaching toxic levels. The agent may also be timed for release after a predetermined delay.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A method of delivering a therapeutic agent to the adventitia of a vessel using a catheter-based microsyringe, comprising:
forming a therapeutic agent into a plurality of microparticles;
dispersing the microparticles throughout a pharmaceutically acceptable liquid carrier to form a therapeutic mixture;
providing a catheter, the catheter including a microsyringe operably attached to an actuator, the microsyringe including a hollow needle in fluid communication with a therapeutic agent delivery conduit, wherein the actuator is operable between an unactuated condition in which the needle is enclosed within the actuator and an actuated condition in which the needle is thrust outward by the actuator;
introducing the catheter into a target area of a vessel;
operating the actuator such that the needle is thrust outward and into a wall of the vessel;
supplying the therapeutic mixture to the therapeutic agent delivery conduit;
delivering the therapeutic mixture through the therapeutic agent delivery conduit to the needle and through the needle into an adventitia of the vessel;
operating the actuator such that the needle is withdrawn from the wall of the vessel and enclosed within the actuator; and
removing the catheter from the vessel.

2. The method of claim 1 wherein the actuator comprises an inflatable balloon.

3. The method of claim 2 wherein operating the actuator such that the needle is thrust outwardly and into a wall of the vessel comprises inflating the balloon.

4. The method of claim 2 wherein operating the actuator such that the needle is withdrawn from the vessel and enclosed within the actuator comprises deflating the balloon.

5. The method of claim 1 wherein forming the therapeutic agent into a plurality of microparticles comprises combining the therapeutic agent with a plurality of microspheres.

6. The method of claim 5 wherein combining the therapeutic agent with a plurality of microspheres comprises encapsulating the therapeutic agent within a plurality of microspheres.

7. The method of claim 5 wherein combining the therapeutic agent with a plurality of microspheres comprises attaching the therapeutic agent to an outer surface of a plurality of microspheres.

8. The method of claim 5 wherein the microspheres comprise one of a biodegradable matrix material and a biocompatible matrix material.

9. The method of claim 8 wherein the matrix material is selected from a group consisting of a biodegradable polymer, a biocompatible polymer, a protein, a polysaccharide, and a lipid.

10. The method of claim 1 wherein forming the therapeutic agent into a plurality of microparticles comprises forming the therapeutic agent into a plurality of dendrimers.

11. The method of claim 1 wherein forming the therapeutic agent into a plurality of microparticles comprises positioning the therapeutic agent as guest molecules within a plurality of dendritic voids formed in a plurality of dendrimers.

12. The method of claim 1 wherein forming the therapeutic agent into a plurality of microparticles comprises forming the therapeutic agent into globules that comprise a discontinuous phase of an emulsion.

13. The method of claim 12 wherein dispersing the microparticles throughout a pharmaceutically acceptable liquid carrier comprises mixing the therapeutic agent with a biocompatible dispersion medium to form an emulsion.

14. The method of claim 13 wherein a portion of the therapeutic agent delivery conduit or the needle is tortuous, and wherein the emulsion is mixed by the resulting turbulence prior to delivery.

15. The method of claim 13 wherein a portion of the therapeutic agent delivery conduit includes at least two channels that empty into a single lumen within the delivery conduit, and wherein the emulsion is mixed by the resulting turbulence prior to delivery.

16. The method of claim 1 wherein forming the therapeutic agent into a plurality of microparticles permits a timed release of the therapeutic agent.

17. The method of claim 1 further comprising:
prior to removing the catheter from the vessel, repositioning the catheter;
operating the actuator such that the needle is thrust outward and into a wall of the vessel;
supplying the therapeutic mixture to the therapeutic agent delivery conduit;
delivering the therapeutic mixture through the therapeutic agent delivery conduit to the needle and through the needle into an adventitia of the vessel; and
operating the actuator such that the needle is withdrawn from the wall of the vessel and enclosed within the actuator.

18. A method of compounding a therapeutic agent for delivery to the adventitia of a vessel using a catheter-based microsyringe, comprising:
forming a therapeutic agent into a plurality of microparticles; and
dispersing the microparticles throughout a liquid carrier suitable for delivery to an adventitia of a vessel.

19. The method of claim 18 wherein forming the therapeutic agent into a plurality of microparticles comprises combining the therapeutic agent with a plurality of microspheres.

20. The method of claim 19 wherein combining the therapeutic agent with a plurality of microspheres comprises encapsulating the therapeutic agent within a plurality of microspheres.

21. The method of claim 19 wherein combining the therapeutic agent with a plurality of microspheres comprises attaching the therapeutic agent to an outer surface of a plurality of microspheres.

22. The method of claim 19 wherein the microspheres comprise one of a biodegradable matrix material and a biocompatible matrix material.

23. The method of claim 22 wherein the matrix material is selected from a group consisting of a biodegradable polymer, a biocompatible polymer, a protein, a polysaccharide, and a lipid.

24. The method of claim 18 wherein forming the therapeutic agent into a plurality of microparticles comprises forming the therapeutic agent into a plurality of dendrimers.

25. The method of claim 18 wherein forming the therapeutic agent into a plurality of microparticles comprises positioning the therapeutic agent as guest molecules within a plurality of dendritic voids formed in a plurality of dendrimers.

26. The method of claim 18 wherein forming the therapeutic agent into a plurality of microparticles comprises forming the therapeutic agent into globules that comprise a discontinuous phase of an emulsion.

27. The method of claim 26 wherein dispersing the microparticles throughout a liquid carrier comprises mixing the therapeutic agent with a biocompatible dispersion medium to form an emulsion.

28. The method of claim 18 wherein forming the therapeutic agent into a plurality of microparticles permits a timed release of the therapeutic agent.
